(19) United States
(12) Patent Application Publication
(10) Pub. No.: US 2026/0285975 A1
(43) Pub. Date: Sep. 24, 2026

(54) TARGETING TIM-3 AND LAG-3 RECEPTORS INDUCED BY CD44+ CD90+ CANCER STEM CELLS IN SMALL CELL LUNG CANCER

(71) Applicant: HACETTEPE UNIVERSITESI, Ankara (TR)

(72) Inventors: Gunes ESENDAGLI, Ankara (TR); Muammer Alper KURSUNEL, Ankara (TR)

(73) Assignee: HACETTEPE UNIVERSITESI, Ankara (TR)

(21) Appl. No.: 19/685,501

(22) Filed: May 22, 2026

Related U.S. Application Data
(62) Division of application No. 17/917,956, filed on Mar. 4, 2021.

(30) Foreign Application Priority Data
Apr. 10, 2020 (TR) 2020/05738

(51) Int. Cl. C07K 16/28; A61P 11/00; A61P 35/00

(52) U.S. Cl. CPC C07K 16/2803; A61P 11/00; A61P 35/00; C07K 16/2827; C07K 2317/31

(57) ABSTRACT

Expression of TIM-3 and LAG-3 co-inhibitory molecules on T cells induced by CD44⁺ CD90⁺ cancer stem cells more than other co-inhibitor molecules/receptors in small cell lung cancer (SCLC). Furthermore, the uses related to targeting TIM-3 and LAG-3 co-inhibitory molecules whose expression in T cells is increased in small cell lung cancer is provided.

### CROSS REFERENCE TO THE RELATED APPLICATIONS

This application is a divisional (DIV) application of non-provisional application Ser. No. 17/917,956 filed on Oct. 10, 2022, which is the national phase entry of International Application No. PCT/TR2021/050192, filed on Mar. 4, 2021, which is based upon and claims priority to Turkish Patent Application No. 2020/05738, filed on Apr. 10, 2020, the entire contents of which are incorporated herein by reference.

### TECHNICAL FIELD

The invention is related to the inhibitors targeting the TIM-3 and LAG-3 co-inhibitory molecules whose expression is increased in T cells in small cell lung cancer, by showing that T cells induced by the (SCLC), CD44+ CD90+ cancer stem cells express TIM-3 and LAG-3 co-inhibitory molecules more than other co-inhibitor molecules/receptors in small cell lung cancer.

### BACKGROUND

Tumors can reshape their microenvironment to escape from the immune system. For this purpose, they use immune regulatory mechanisms that are also used by normal cells to prevent immunopathologies. Small cell lung cancer (SCLC) is associated with aggressive tumor growth, early spread and distant metastasis tendency. Although chemotherapy responses begin optimally, therapy resistance develops in a short time. These features of SCLC point to the concept of cancer stem cell (CSC). Recent studies describe candidate stem cell populations in SCLC. Although mesenchymal and cancer stem cells are stated to be immune-tolerogenic, there is not much information about the results of the interaction between SCLC stem cells and immune cells.

Small cell lung cancers are currently being treated with platinum-based chemotherapeutics. Despite a very good response in the first place, these patients develop drug resistance very quickly, and for this reason, the survival time of the patients is very short. Alternatively, popular immunotherapy approaches are tried for different cancers. The prominent applications here are also anti-PD-1, anti-PD-L1 and anti-CTLA-4 treatments and blocking of common T cell co-inhibitors.

Due to the low incidence of SCLC, almost all of the molecular studies in lung cancer and the studies describing the relationship of lung cancer with the immune system come from non-small cell lung cancer (NSCLC).

This causes the relationship between SCLC and the immune system to be understood poorly. As a result, anti-PD-1, anti-PD-L1 and anti-CTLA-4 trials, which have been tried extensively for almost every cancer, including NSCLC in the clinic, have moved into the clinical phase considering the possibility of benefiting from them. However, the results obtained show that the desired success could not be achieved. This demonstrates the importance of correctly targeted immunotherapy approaches with an understanding of SCLC immunology.

In the prior art, the combination blocking of PD-1 and LAG-3 co-inhibitors is mentioned. In this prior art document, inhibitors were used on B16 melanoma cells, MC38 colon adenocarcinoma cells, Sa1N fibrocarcinoma cells. In another article, the relationship of LAG-3 protein expression with PD-1, PD-L1 and tumor-infiltrating lymphocytes (TILs) in non-small cell lung cancer (NSCLC) is examined. It is known that receptors such as PD-1, CTLA-4, TIM-3 and LAG-3 are expressed in T cells of patients with cancer and negatively affect T cell responses. For this reason, it is possible to give them both separately and in combination in different cancers. Prior art documents mention such uses in different cancers. However, none of the aforementioned documents contain data specific to small cell lung cancer (which is different from non-small cell lung cancer and should be considered separately from this lung cancer subtype).

PD-1 or CTLA-4 receptors targeted in the prior art are insufficient in the treatment of small cell lung cancer. Platinum-based chemotherapeutics, another alternative, are intended for all cancer cells and cancer stem cell targeting approaches are not applied in clinical trials because there is insufficient information about the results of the interaction between SCLC stem cells and immune cells.

Therefore, there is a need to define the interaction between SCLC cells and T cells, including the stem cell population, and to identify new target receptors-molecules by investigating the immunomodulatory mechanisms of SCLC.

### SUMMARY

The invention is related to the inhibitors which target T cell co-inhibitory receptors (TIM-3, LAG-3) induced by CD44+ CD90+ cancer stem cells (including their adaptive resistance capacity) or small cell lung cancer cells (particularly CD44+ CD90+ cancer stem cells) in the treatment of small cell lung cancer.

The description of the inhibitor in the specification and claims will comprise the following molecules or compositions singularly, as well as the combination of the same:
Small molecule inhibitors targeting LAG-3 or TIM-3,
LAG-3 or TIM-3 antagonist molecules,
Compositions for silencing the gene encoding the LAG-3 or TIM-3 receptor in T cells,
bispecific antibodies
antibody-mediated nanoparticles

The purpose of the invention is to define a treatment method with inhibitors targeting LAG-3 and/or TIM-3 by revealing the interaction between SCLC cells and T cells, including the stem cell population, and the immune regulatory mechanisms of SCLC cancer.

The results obtained with the invention show that small cell lung cancer cells (especially cancer stem cells) make T cells susceptible to co-inhibition while using molecules such as TIM-3 and LAG-3 instead of current treatment targets PD-1 or CTLA-4. uses. Therefore, it can be seen that the treatments targeting these molecules may play a more critical role in targeting small cell lung cancers.

It is put forth by the invention that PD-1/PD-L1 treatments that are tried for cancer will not be very successful because targeting TIM-3 and especially LAG-3 in small cell lung cancer will be more meaningful. These findings are completely novel and are not subject to any literature or patent applications.

In addition, besides targeting LAG-3 alone, combined treatment with CD44, TIM-3, PD-1 or PD-L1 is also possible.

With the invention, it is ensured that small cell lung cancer cells targeted with the existing chemotherapy and immunotherapy approaches are developed with more accurate and specific targeting.

### BRIEF DESCRIPTION OF THE DRAWINGS

FIGS. 1A-1B: Heatmaps showing the mesenchymal associated (FIG. 1A) and stem-cell related genes (FIG. 1B), increased at least 24 times in mesenchymal stem cells (MSC) pared to alveolar type II cells (AEC type II)

FIGS. 2A-2D: PD-1, CTLA-4, TIM-3 and LAG-3 expression (*p<0.05, **p<0.01, ***p<10⁻³, p<10⁻⁴, n≥4) on the CD8⁺ T cells in the PBMC cells co-cultured with SCLC subclones in the presence of αCD3 mAb (25 ng/ml) stimulation after 96 hours.

FIGS. 3A-3D: PD-1, CTLA-4, TIM-3 and LAG-3 expression (*p<0.05, **p<0.01, ***p<10⁻³, ****p<10⁻⁴, n≥4) on the CD4⁺ T cells in the PBMC cells co-cultured with SCLC subclones in the presence of αCD3 mAb (25 ng/ml) stimulation after 96 hours.

FIG. 4: Immunohistochemistry staining of co-inhibitor molecules in tumor infiltrated lymph nodes (tumor-draining lymph nodes) of SCLC patients. Representative sections of paraffin-embedded samples (40× scale) showing LAG-3, PD-L1, PD-1 and TIM-3.

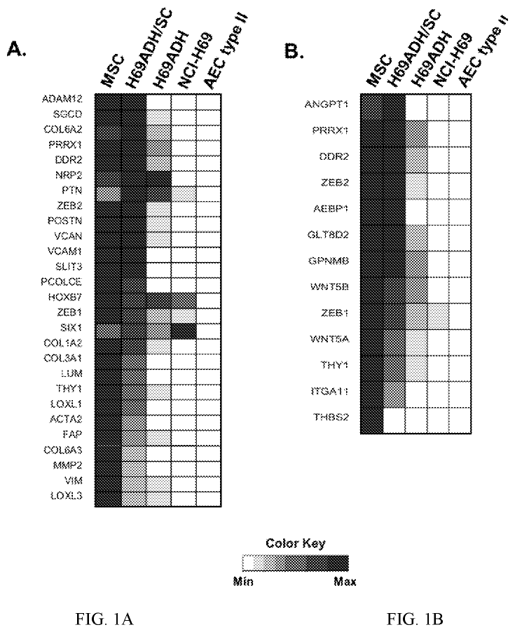

### DETAILED DESCRIPTION OF THE EMBODIMENTS

It is revealed by the invention that CD44+ CD90+ cells in small cell lung cancer have stem cell character and have the highest immunomodulation capacity in these cells.

The description of the inhibitor in the specification and claims will comprise the following molecules or compositions singularly, as well as the combination of the same:
Small molecule inhibitors targeting LAG-3 or TIM-3,
LAG-3 or TIM-3 antagonist molecules,
Compositions for silencing the gene encoding the LAG-3 or TIM-3 receptor in T cells,
bispecific antibodies
antibody-mediated nanoparticles

Peripheral blood mononuclear cells (PBMC) obtained from healthy volunteers are cultured with the subclones of SCLC cells in the presence of αCD3 mAb, and expression, proliferation, cytokine production, and expression of inhibitory co-receptors of the T cell activation markers are determined in the T cells. PDL1 and PD-L2 expression in SCLC cells is determined in PBMC co-cultures with or without IFN-γ stimulation or IFN-γ blockage, and the effects of PD-L1 expression on the CD8⁺ T cell proliferation and T cell cytotoxicity of SCLC cells are shown. Functional capacity of TIM-3⁺ LAG-3⁺ and TIM-3⁻LAG-3⁻ CD8⁺ T cells obtained from SCLC co-cultures is evaluated in vitro. SCLC cells allow and even support T cell activation in a way to be more pronounced by stem cell-like SCLC cells. In addition, stem cell-like SCLC cells exhibit adaptive resistance properties by inducing PD-L1 and PD-L2 when exposed to IFN-γ and activated PBMCs. That being said, the presence of PD-L1 on these cells does not lead to the proliferation of cytotoxic T lymphocytes and the decreased susceptibility by the SCLC cells to T-cell cytotoxicity.

Alternatively, the expression of co-inhibitory receptors on T cells is determined to be more susceptible to immunosuppression in the presence of SCLC cells since they expose T cells to inhibitory signals. To summarize, in the present invention, the effect of stem cell-like SCLC cells in the regulation of immune modulation by inducing inhibitory co-stimulatory molecules in the tumor microenvironment and their use in potential immunotherapy approaches are discussed.

### Increased Expression of Co-Inhibitory Receptors on T Cells Induced by SCLC Subclones

In the invention, SCLC cell lines (NCI-H82, NCI-H69, SCLC-21) grown as suspension, subclones attached to stem cell-like plastic isolated from NCI-H82 and NCI-H69, and CD44⁺CD90 subclone purified from the subclone of NCI-H69 are used. PBMC cells are used as a control group and SCLC cell lines and PBMCs are co-cultured, thereby the change of the PD-1, CTLA-4, TIM-3 ve LAG-3 co-inhibitors on CD4⁺ and CD8⁺ T cells are observed.

The cells in the PBMC: SCLC co-culture which is kept in the culture environment for 96 hours are collected afterward, marked with fluorescent-carrying monoclonal antibodies marking both the genealogy markers (CD4, CD8) and co-inhibitor receptors (PD-1, CTLA-4, TIM-3, LAG-3), and the flow is analyzed in the cytometry device.

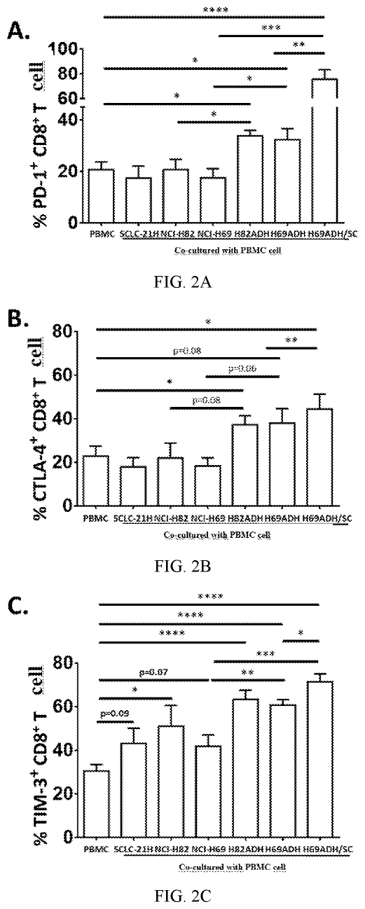

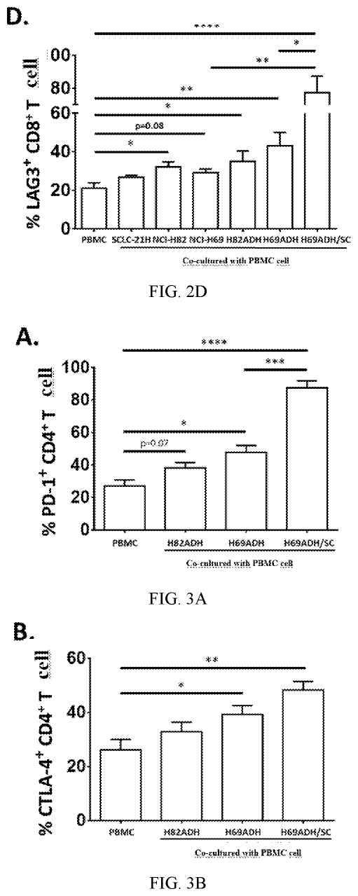

Alongside the fact that the PD-L1 and PD-L2 expression of the SCLC cells, particularly the subclones attached to the plastic, is increased (upregulation) when compared to the inflammatory cytokines such as activated T cells/IFN-γ, as shown in FIGS. 2A-2D, stimulation of the PBMCs with αCD3 (25 ng/ml) for 96 hours provides for limited PD-1 expression (20.68%+2.96%). As a result of the co-culture of PBMCs with SCLC cells, subpopulations of SCLC increase the PD-1 expressing CD8⁺ T cell ratio in PBMCs on the levels of 33.74%±2.16 when co-cultured with H82ADH, 32.28%±4.27 when co-cultured with H69ADH, and 75.64%±7.69 when co-cultured with H69ADH/SC (FIG. 2A).

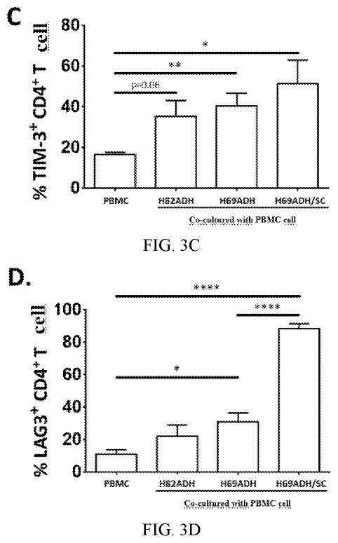

CD4⁺ T cells expressing PD-1 in PBMCs stimulated with H69ADH/SC stem cells in the presence of αCD3 for 96 hours reach 87.35%±4.31. Similarly, when PBMCs are co-cultured with other stem cell-related subclones, an increase is observed in the ratio of CD4⁺ T cells expressing PD-1 (FIG. 3A).

When PBMCs are co-cultured with SCLC cells, the ratio of T cells expressing CTLA-4 in PBMCs appears to be low, similar to PD-1⁺ T cells. While the ratio of CTLA-4⁺CD8⁺ cells is 22.88%±4.66 in the control group, this ratio does not change more than 5% in PBMCs co-cultured with parental SCLC cell lines. However, it is seen that CTLA-4-positivity increases to 44.47%±6.882 in co-cultures where H69ADH/SC is used (FIG. 2B). Also, the ratio of CD4⁺ T cells expressing CTLA-4 increases up to 50% in co-cultures using H69ADH/SC (FIG. 3B).

More significantly than PD-1 and CTLA-4 co-inhibitory receptors, the percentage of TIM-3-expressing CD8⁺ T cells increases significantly in all SCLC subclones-PBMC co-cultures compared to the PBMC control group. The rate of these increases (TIM-3⁺CD8⁺) is 30.55%±2.86 in the PBMC control group in FIG. 2C, and it is seen as 41.88%±5.27 in NCI-H69 co-cultures and 71.42%±3.63 in H69ADH/SC co-cultures. In CD4⁺ T cells, as shown in FIG. 3C, the increase in TIM-3 expression in H69ADH/SC co-cultures is almost 3 times higher than in the control PBMC group.

In addition to TIM-3, the ratio of CD8⁺ T cells expressing LAG-3 also increases significantly in SCLC co-cultures compared to the PBMC control group. While 21.06%±2.76 of LAG-3⁺ CD8⁺ T cells were observed in the control PBMC group, the rate increases to 26.73%±0.99 and 43.05%±6.85 in PBMCs co-cultured with subclonal SCLC cell lines grown in suspension or attached to plastic. More strikingly, the percentage of LAG-3⁺ CD8⁺ T cells increases to 77.35%±10.03 when PBMCs interact with H69ADH/SC (FIG. 2D). Likewise, the amount of CD4⁺ T cells expressing LAG-3 appears to be increased by 88.38%±2.89 (FIG. 3D). Since SCLC cell subclones are more effective in promoting T cell activation and inducing inhibitory receptors on CD8⁺ T cells, inhibitory receptor expression on CD4⁺ T cells was only been investigated in co-cultures with SCLC subclones and its effect was demonstrated (FIGS. 3A-3D). These results show that in vitro, TIM-3 and LAG-3 induction capacities in T cells, especially stem cell subclones, of SCLC cells are more significant than PD-1 and CTLA-4.

Since SCLC cells were found to increase the expression of PD-1, TIM-3 and LAG-3 on T cells in the invention, the presence of these markers in SCLC patient tissues was evaluated. For this purpose, the primary tumor sample from SCLC patients is initially marked with αCD3. However, it is observed that CD3⁺ T cells surround the tumor tissue but do not infiltrate into the tumor. This shows that the T cells do not interact much with SCLC cancer tumor cells in the primary tumor. Alternatively, thirteen lymph node samples from SCLC patients are labeled with monoclonal antibodies PD-1, PD-L1, TIM-3 and LAG-3. It is observed that metastatic foci are evident in all metastatic lymph nodes.

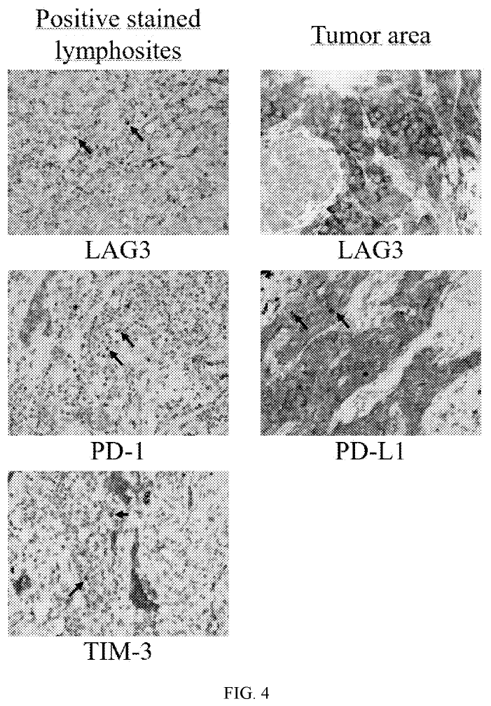

Immunohistochemistry staining of co-inhibitor molecules (LAG-3, PD-L1, PD-1 and TIM-3) on tumor infiltrated lymph nodes (tumor-draining lymph nodes) of SCLC patients is shown in FIG. 4. Samples of 4 μm thickness are taken from paraffin-embedded tissues and examined (40× scale). After the slides are incubated with primary antibodies (anti-PD-L1, 20 min 1/100 dilution; anti-PD-1, 20 min 1/250 dilution; anti-TIM-3, 10 min 1/1500 dilution; anti-LAG-3, 20 min 1/625 dilution), following the washing step, they are set left incubation with secondary antibodies and incubated with streptavidin-biotin complex. After staining with hematoxylin, target molecules in the sample samples are viewed under a conventional light microscope. Similar to primary tumors, immune cells are mainly located around metastatic foci but with more infiltrating cells. It is seen that most of the lymph node samples shown in FIG. 4 contain lymphocytes expressing LAG-3 (Table 1). In addition, it is observed that LAG-3 is expressed focally on some metastatic tumor cells in tumor infiltrated lymph nodes.

TABLE 1
Analysis of the expression frequency of inhibitor molecules
in tumor-draining lymph nodes of SCLC patients

| Inhibitory molecule | Positively marked samples |
|---|---|
| LAG-3⁺ | 10/13 |
| PD-1⁺ | 4/13 |
| TIM-3⁺ | 2/13 |
| PD-L1⁺ | 2/13 |

CD90 is commonly expressed in all SCLC cell subclones. Therefore, CD90 percentage is not seen as a discriminating parameter for identifying stem cell-like cancer cells in the SCLC cell population. However, RNAseq analysis shows that CD90 expression is increased from NCI-H69 to H69ADH. There is even a separate CD44⁺ CD90^{high}subpopulation in the H69ADH cell line and this population is isolated as H69ADH/SC.

CD44 expression is restricted only in subclonal SCLC cell lines growing in suspension and attached to plastic. CD44⁺CD90^{high} H69ADH/SC cells display induced mesenchymal and stem cells specific character. In addition, it is known that urokinase plasminogen activator receptor (uPAR/CD87) expression is associated with invasive phenotypes and reduced survival rate. Since CD87 regulates extracellular matrix degradation, it is also involved in cell migration and invasion; Therefore, SCLC cells expressing CD87 show induced stem cell character and multi-drug resistance, high clonogenic activity and co-expression of cancer stem cell markers CD44 and MDR1. Transcriptomic data demonstrate that the subclones (H82ADH, H69ADH and H69ADH/SC) attached to the SCLC mesenchymal stem cell-like plastic shown in the invention have CD87 expression, but no CD87 expression in the suspension growing SCLC cell lines (CDLC-21H, NCI-H82 and NCI-H69).

In summary, it is thought that CD44 surface protein and CD87 mRNA can be used to differentiate/identify cancer stem cells in SCLC cell lines together with the high population of CD90^{high}. Although SCLC subclones attached to plastic show low levels of CD44 ve CD90^{high} cell percentages, they are capable of in vitro differentiation and in vivo tumor initiation associated with cancer stem cells. Therefore, SCLC subclones attached to plastic have a character/trait that falls between the epithelial and the stem cell fraction.

SCLC expresses TIM-3 and LAG-3 at a high rate, especially in metastatic foci, much more significant than the induction of PD-1 and CTLA-4 in CD4+ve CD8+ T cells by its subclones attached to the plastic. At the same time, LAG-3 is the most frequently expressed check-point receptor on lymphocytes in metastatic lymph nodes of SCLC patients. Therefore, targeting of LAG-3 is thought to be an effective means of checkpoint blockage immune intervention treatments.

With the invention;
In small cell lung cancer, the capacity to develop adaptive resistance to inflammatory cytokines such as IFN-γ, and PD-L1 expression of mesenchymal character SCLC cells were found to be higher in parallel with the stem cell feature. Therefore, therapeutic methods in small cell lung cancer, including mesenchymal markers such as CD44, CD90, Vimentin, as well as targeting the PD-L1 molecule (e.g. bispecific antibodies and a mesenchymal marker, PD-L1 targeting antibodies),
Methods involving supporting T cell activation by targeting co-inhibitor molecules such as TIM-3 and LAG-3, whose expression increases in T cells due to the immunomodulation capacity of SCLC cells, cancer stem cells being in the first place,
Since it has been determined that lung cancer stem cells can induce the expression of LAG-3 and TIM-3 and can express it themselves to some extent, methods involving targeting tumor and metastasis in small cell lung cancer in LAG-3 and TIM-3 targeted therapies are preserved.

Since it has been shown for the first time with the results mentioned in the specification that SCLC stem cells can express LAG-3 also in the metastatic focus, LAG-3 targeting to be applied with inhibitors is also critical in terms of targeting stem cells which are found in metastasis foci and which are thought to manage metastasis.

The description of the inhibitor in the specification and claims will comprise the following molecules or compositions singularly, as well as the combination of the same:
Small molecule inhibitors targeting LAG-3 or TIM-3,
LAG-3 or TIM-3 antagonist molecules,
Compositions for silencing the gene encoding the LAG-3 or TIM-3 receptor in T cells,
bispecific antibodies
antibody-mediated nanoparticles

With the invention, the use of LAG-3 or TIM-3 inhibitors in metastatic small cell lung cancer treatment is preserved.

What is claimed is:

1. An in vitro method, comprising:
culturing peripheral blood mononuclear cells (PBMC) obtained from healthy volunteers with small cell lung cancer (SCLC) cells or subclones of SCLC cells in the presence of αCD3 mAb;
maintaining the PBMC: SCLC co-culture in the culture environment for 96 hours;
collecting cells in the PBMC: SCLC co-culture after the 96 hours and staining the collected cells with fluorescently labeled monoclonal antibodies to lineage markers CD4 and CD8 and co-inhibitory receptors PD-1, CTLA-4, TIM-3, and LAG-3; and
analyzing the stained cells by flow cytometry.

2. The in vitro method according to claim 1, wherein the SCLC cells comprise subclones attached to stem cell-like plastic isolated from NCI-H82 and NCI-H69.

3. An immunohistochemistry staining method, comprising:
taking samples of 4 μm thickness from paraffin-embedded tissues of tumor infiltrated lymph nodes of small cell lung cancer (SCLC) patients;
incubating slides with primary antibodies comprising anti-PD-L1, anti-PD-1, anti-TIM-3, and anti-LAG-3;
following a washing step, incubating the slides with secondary antibodies and with a streptavidin-biotin complex; and
after staining with hematoxylin, viewing target molecules under a conventional light microscope at 40× scale.
